(19) United States
(12) Patent Application Publication
(10) Pub. No.: US 2021/0383919 A1
(43) Pub. Date: Dec. 9, 2021

(54) MEDICAL GAS MANAGEMENT SYSTEM

(71) Applicant: Jaymes Christian Provost, Pompano Beach, FL (US)

(72) Inventor: Jaymes Christian Provost, Pompano Beach, FL (US)

(21) Appl. No.: 16/891,163

(22) Filed: Jun. 3, 2020

(51) Int. Cl. G16H 40/40; G16H 40/20; G06Q 10/06; G06Q 10/00; G16H 70/20; G06Q 50/28; G06Q 30/00; G06Q 20/04; G06Q 10/10

(52) U.S. Cl. CPC G16H 40/40; G16H 40/20; G06Q 10/06314; G06Q 10/20; G16H 70/20; A61M 16/0063; G06Q 30/018; G06Q 10/06375; G06Q 20/045; G06Q 10/1097; G06Q 10/0635; G06Q 50/28

(57) ABSTRACT

A system for managing medical gasses system is disclosed, comprising a database configured to store a plurality of asset information. A user dashboard is provided on an interface of a computing device in operable communication with the database, the user dashboard to provide an interface for engaging with the asset information. A calendar module to receive scheduling information corresponding to the plurality of asset information. An asset manager receives the plurality of asset information and generate a heat map of the plurality of assets to facilitate the management of the plurality of assets.

### TECHNICAL FIELD

The embodiments generally relate to inventory management systems and, more particularly, relate to systems for the management of the medical gas supply chain.

### BACKGROUND

In modern medical facilities and scientific research facilities, there is a need to supply medical gases to various points of use including, for example, patient rooms, operating rooms, examination rooms, and the like. It is common for the facilities to have a central storage point from which the gasses are distributed to various locations via a network of conduits. Gasses are stored in pressurized gas cylinders, which can range in size from 5 KG to 500 KG and larger. The supply of gas may be monitored by a centralized system which measures variables such as gas pressure or liquid level at the storage container. Some systems automate the ordering process by initiating an order when gasses reach a low threshold level.

For consumers and in order to control costs, it may be preferable to maintain only a necessary amount of raw materials (e.g., gasses) to ensure continuity of operations. Thus, inventory management is important for maintaining adequate profitability, safety, and efficiency.

For providers, automated systems exist for the ordering and distribution of gas; however, these systems do not manage inventory of the deliverables either at the gas company facility or at the point of use. In such, managing the location and status of gas cylinders after delivery is typically done by hand, resulting in inaccuracies and inefficiencies when reporting data. Historically, some aspects of the scheduling of inventory delivery, retrieval, inspections, installations, and maintenance/servicing has been performed manually, resulting in efficiencies for the consumer and provider alike.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments provided herein relate to a system for managing medical gasses system, comprising a database configured to store a plurality of asset information. A user dashboard is provided on an interface of a computing device in operable communication with the database, the user dashboard to provide an interface for engaging with the asset information. A calendar module is configured to receive scheduling information corresponding to the plurality of asset information. An asset manager receives the plurality of asset information and generates a heat map of the plurality of assets to facilitate the management of the plurality of assets. The system may be used to generate a heat map to facilitate asset tracking related to the delivery, consumption, and retrieval of gasses and/or liquids.

In one aspect, the plurality of asset information comprises at least one of the following: customer information, equipment information, inventory information, asset location information, service requests, preventative maintenance information, risk ratings, code references, and asset tag tracking information.

In one aspect, the system comprises a plan of action system to receive the plurality of asset information and identify a deficiency with one or more assets.

In one aspect, the plan of action system comprises a compliance engine to identify a deficiency in compliance.

In one aspect, the plan of action system further comprises an action engine to identify an action to correct the deficiency in compliance.

In one aspect, the plan of action system further comprises a quote generator to generate a quote corresponding to the action to correct the deficiency in compliance.

In one aspect, a service ticket generator is configured to generate a service ticket corresponding to the quote.

In one aspect, the quote generator is in operable communication with the calendar module to monitor a status of the quote.

In one aspect, the quote generator is in operable communication with the calendar module to schedule one or more reminders corresponding to the quote and transmit the reminders to at least one user.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure, according to some embodiments;

FIG. 2 illustrates a block diagram of the server engine and modules, according to some embodiments;

FIG. 3 illustrates a block diagram of the plan of action system, according to some embodiments;

FIG. 4 illustrates a block diagram of the preventative maintenance engine, according to some embodiments;

FIG. 5 illustrates a block diagram of the manufacturer assembly system, according to some embodiments;

FIG. 6 illustrates a block diagram of the asset manager, according to some embodiments;

FIG. 7 illustrates a screenshot of the user dashboard interface, according to some embodiments;

FIG. 8 illustrates a screenshot of the calendar interface, according to some embodiments; and

FIG. 9 illustrates a block diagram of the heat map generator, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, the term “user” may relate to any person interacting with the various features of the system provided herein and may include administrative users, users at the provider location, service providers, consumers of the deliverable item (e.g., medical professionals utilizing the gas), inventory management professionals (both at the provider storage facility and the consumer storage facility), and the like.

As used herein, the term “asset” refers to items associated with medical gasses and its delivery. Assets may include gas cylinders, raw materials (gasses and/or liquids), supply materials (conduits, delivery systems, monitoring systems, etc.), alarm systems, delivery vehicles, delivery personnel, and other personnel, products, materials, etc. associated with the delivery, consumption, and retrieval of medical gasses and assets thereof.

In general, the system described herein relates to a computing system to manage the inventory, delivery, consumption, servicing, inspection, and installation of systems related to medical gasses. The system allows for medical gas providers (referred to herein as “the provider”) and medical gas consumers (referred to herein as “the consumer”) to manage various aspects of the medical gas delivery process. The embodiments may be used for inventory management, raw materials tracking, and to prevent downtime associated with running out of a particular process gas. The system may also be utilized to provide an interlock to prevent a cylinder of the wrong type of gas from being installed at particular location. Further, the host computer of the automated gas cylinder monitoring system of the present invention is programmed to calculate and display a cylinder replacement schedule based on information transmitted to the host computer from the read/write device.

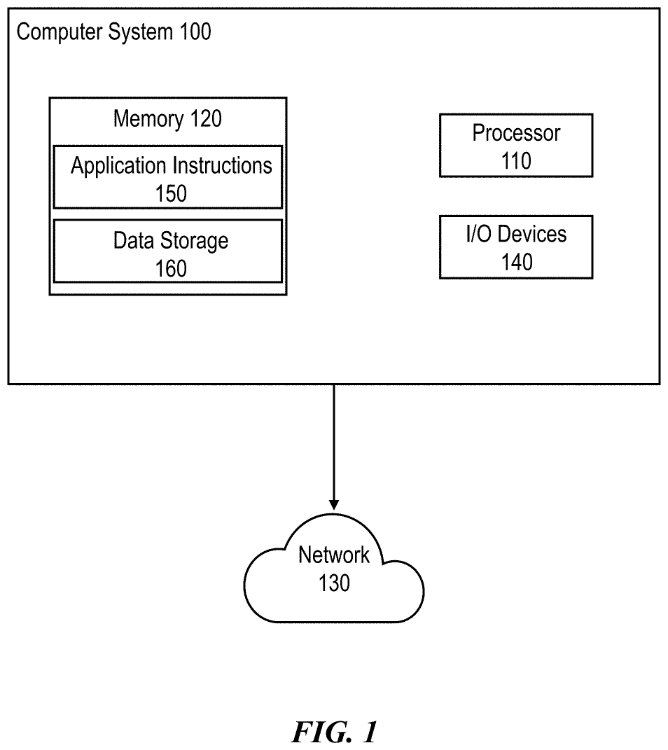

FIG. 1 illustrates a computer system 100, which may be utilized to execute the processes described herein. The computer system 100 is comprised of a standalone computer or mobile computing device, a mainframe computer system, a workstation, a network computer, a desktop computer, a laptop, or the like. The computer system 100 includes one or more processors 110 coupled to a memory 120 via an input/output (I/O) interface. Computer system 100 may further include a network interface to communicate with the network 130. One or more input/output (I/O) devices 140, such as video device(s) (e.g., a camera), audio device(s), and display(s) are in operable communication with the computer system 100. In some embodiments, similar I/O devices 140 may be separate from computer system 100 and may interact with one or more nodes of the computer system 100 through a wired or wireless connection, such as over a network interface.

Processors 110 suitable for the execution of a computer program include both general and special purpose microprocessors and any one or more processors of any digital computing device. The processor 110 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 100 and other devices attached to a network 130, such as other computer systems, or between nodes of the computer system 100. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

The memory 120 may include application instructions 150, configured to implement certain embodiments described herein, and a database 160, comprising various data accessible by the application instructions 150. In one embodiment, the application instructions 150 may include software elements corresponding to one or more of the various embodiments described herein. For example, application instructions 150 may be implemented in various embodiments using any desired programming language, scripting language, or combination of programming languages and/or scripting languages (e.g., C, C++, C#, JAVA®, JAVASCRIPT®, PERL®, etc.).

In one embodiment of the invention, the memory 120 or data storage device 160 associated with gas cylinders comprises a means for receiving and storing information contained on an inventory control collar or ledger provided on the gas cylinder. A pressure gauge may be in operable communication with the system 100 for monitoring the pressure in the cylinder. The inventory control collar includes a resident memory that is capable of storing such general inventory control information as a cylinder identification number, cylinder serial number, owner name and site, product code, product material, DOT hazard classification, content fill level, tare weight, content level during use, fill site and date, and shelf life. Shipping information that may also be stored includes the origin point (including company name, site and code), shipping manifest number, shipping date, destination point (including company name and site), destination order number and product, destination receiving date, and by whom it was received. Transfer information may also be stored and include the transfer point (including company name and site), manifest number, destination point (including company name and site), order number, destination receiving data, and by whom it was received. Further, certification data may be stored and include record number, certifier, analysis, and specification limits. The memory 120 may also be used to record additional manufacturing information such as upset conditions, process steps and operator notations. All of the above listed information is not necessarily stored in the resident memory of the inventory control collar. It may be desirable in some cases to store certain information in the computer system 100, both at the consumer and provider levels.

The steps and actions of the computer system 100 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

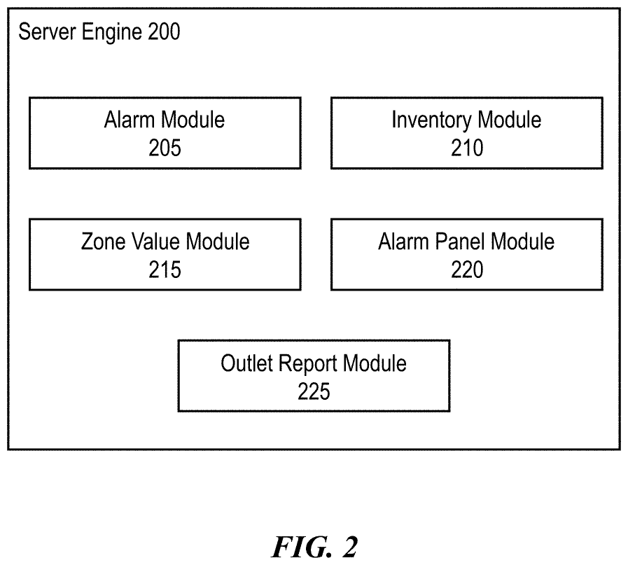

FIG. 2 illustrates a block diagram including a server engine 200 and modules to perform the various functionalities of the embodiments described herein. An alarm module 205 receives models, types, code references, asset tag tracking information, service requests, risk ratings, and similar information which is stored in the database. The alarm module 205 may determine threshold values for each information type stored in the system and generate an output signal if a threshold is exceeded, reached, or approached to alert the user of a service need, deficiency, and the like. An inventory module 210 allows the system to receive input from one or more users related to gas cylinder and cylinder content information. For example, the inventory module 210 receives equipment details, purity reports, images, code references, asset tag tracking information, service requests, and risk ratings. A zone value module 215 receives zone valve box service details, gas type(s), function status (e.g., pass or fail), labeling status, cover status, code references, asset tag tracking, service requests, and risk ratings. An alarm panel module 220 receives an area alarm service status and details thereof, gas types, audible alarm function status, visual alarm functions status, code references, asset tag tracking, service requests, and risk ratings. An outlet report module 225 receives outlet type(s), outlet concentration readings, outlet flow readings, outlet images, manufacture part information, serial number(s), risk ratings, code references, asset tag tracking, and service requests.

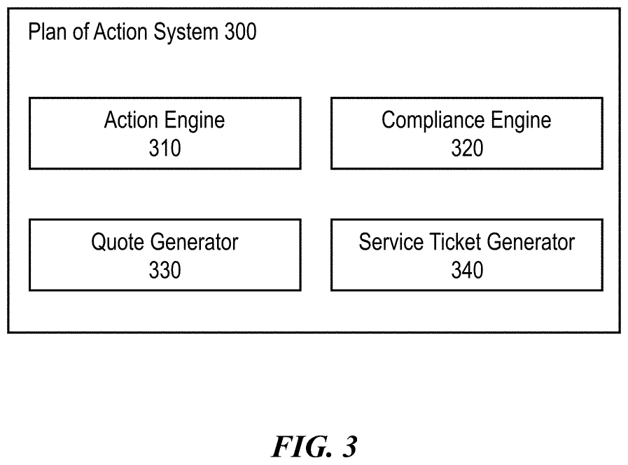

FIG. 3 illustrates a block diagram of the plan of action system 300 comprising an action engine 310 to receive information from the server engine 200 and information output by the modules thereof to provide a plan of action or output when a threshold value is reached, when a service is requested, when a risk rating indicates a deficiency, or when information is required from the consumer. A compliance engine 320 determines if medical gas equipment, design, or materials are out of compliance and allows for code reference, including a keyword search, to be utilized. The action engine 310 may determine an appropriate plan of action to correct a deficiency in compliance. A quote generator 330 generates a quote using a part number(s) and serial number(s) and service information to generate a quote for the part, service, or the like, which are needed to correct a deficiency. A service ticket generator 340 generates a service ticket to track service requests, report a service request or the completion thereof, and the like.

In some embodiments, the quote generator 330 provides a means for transmitting a quote and monitoring the quote status when delivered to the consumer. The quote generator 330 may schedule reminders for following up on a quote following its transmission to the consumer or to remind the provider that a quote is in progress.

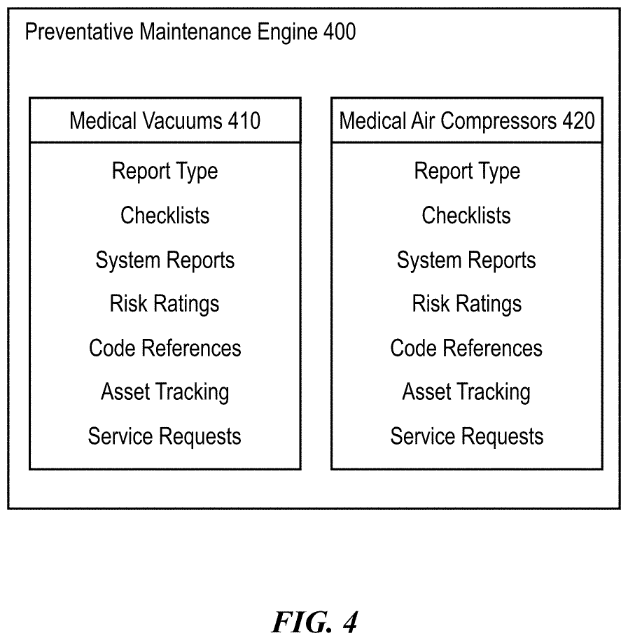

FIG. 4 illustrates a preventative maintenance engine 400 to generate reports related to medical vacuums 410, medical air compressors 420 and similar systems. The medical air compressors 420 report may include information including a report type, checklists, system reports, risk ratings, code references, asset tracking, and service requests. Similarly, the medical vacuums 410 report may include information including a report type, checklists, system reports, risk ratings, code references, asset tracking, and service requests.

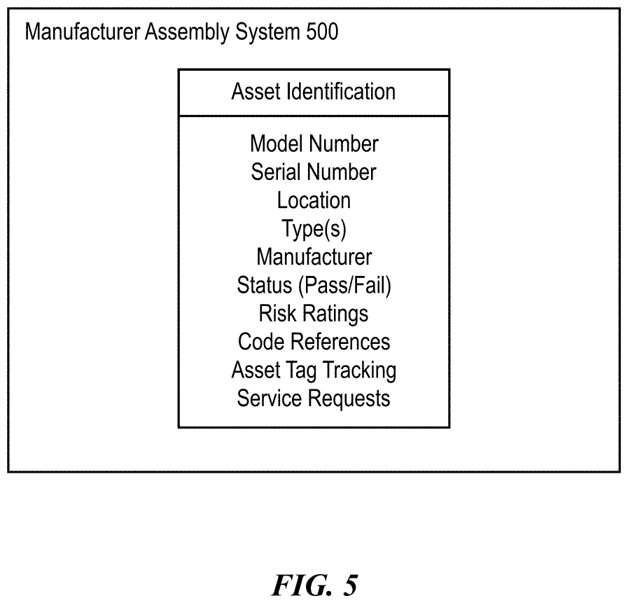

In reference to FIG. 5, a manufacturer assembly system 500 includes information related to various equipment in communication with the medical gas system including nonstationary booms, articulating assemblies, connectors, conduit, etc. The manufacturer assembly system 500 adds manufacturer assembly model/serial numbers (or similar identifying features), locations, types, manufacturer, status information (e.g., pass or fail), risk ratings, code references, asset tag tracking, and service requests.

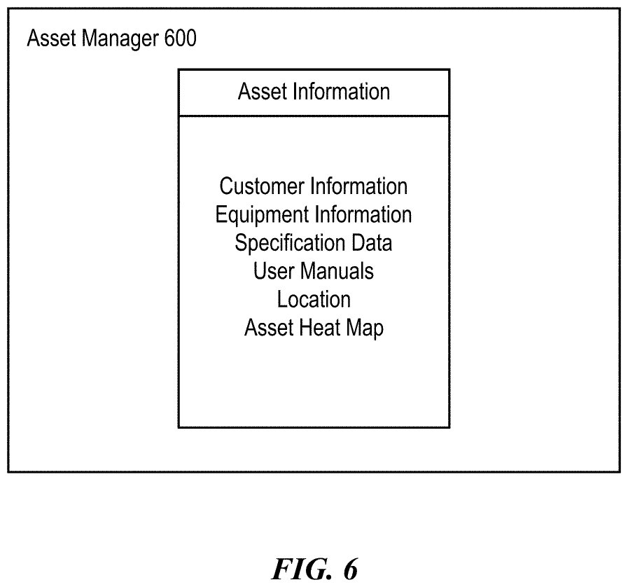

FIG. 6 provides an asset manager 600 to allow the system and users thereof to manage assets (e.g., medical gas systems, cylinders, and gasses). The user may add customer information, equipment information, specification information, user manuals, location information, and generate a heatmap of assets (i.e., inventory of medical gas equipment, cylinders, gas levels, etc.) which are deployed to consumers.

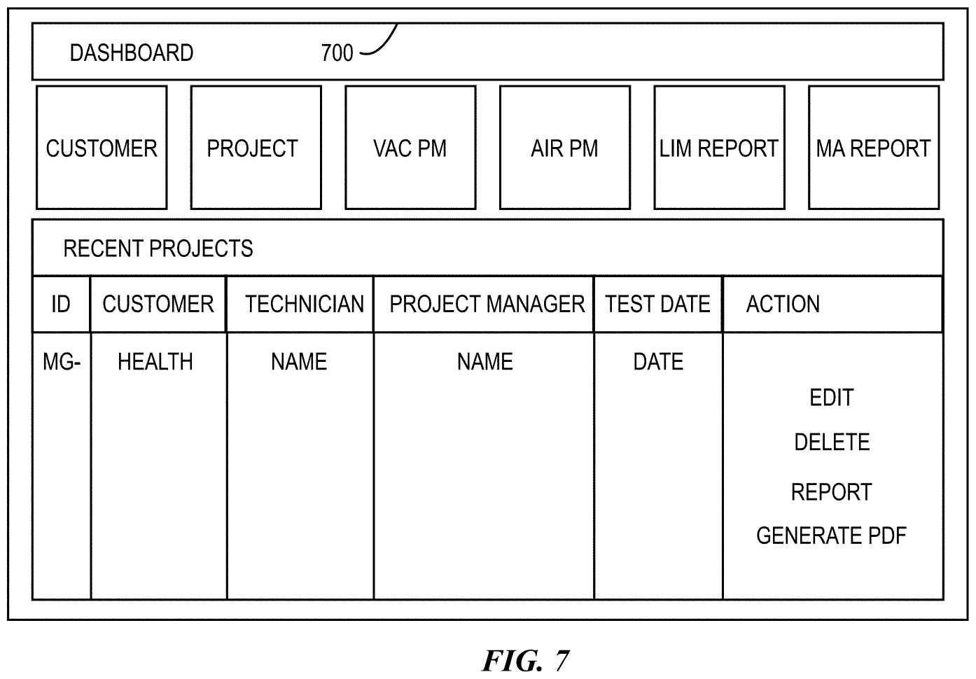

FIG. 7 illustrates a user dashboard 700 which may be provided to the provider computing system and/or consumer computing system to permit the user to interface with the various functionalities of the medical gas management system provided herein. The dashboard 700 provides a plurality of selectable tabs including tabs related to customers, projects, vacuum preventative maintenance, air compressor preventative maintenance, and various reports. A projects interface provides project identifiers, customer information, technician(s) associated with the project, project managers associated with the project, test dates, and actions (including functions to edit, delete, view and generate reports related to the project).

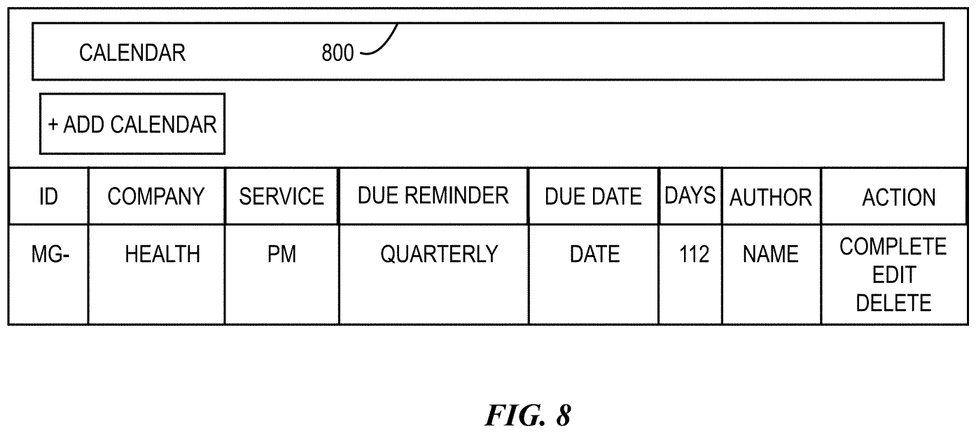

FIG. 8 illustrates a calendar interface 800. To generate a calendar item, the user selects a customer, service, date, and scheduling frequency (weekly, quarterly, bi-annually, annually, etc.) for a project. The calendar interface 800 comprises information related to project identifiers, company information, service information, scheduling frequency (due date reminders), due dates, author of the calendar entry, and actions (including status, edit features, delete functions, and activate/deactivate functionalities).

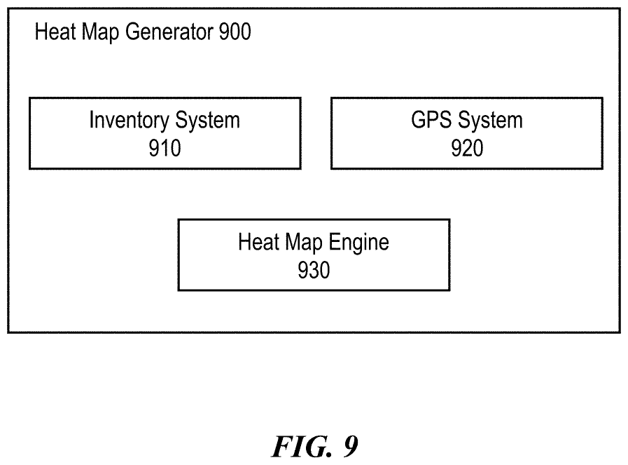

FIG. 9 illustrates a block diagram of a heat map generator 900 comprising an inventory system 910 to receive inventory from the provider, consumer manufacturer, and the like. The inventory module 210 determines an amount of raw material, a number of cylinders, and the like. A GPS system 920 provides location information associated with each asset including the location the asset was delivered, the location the asset is stored (prior to or following delivery), and the like. The inventory system 910 and GPS system 920 output information to a heat map engine 930 to generate a heat map of assets. The heat map generator 900 provides an interface for easily viewing and managing assets.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A system for managing medical gasses systems, comprising:
a database configured to store a plurality of asset information;
a user dashboard provided on an interface of a computing device in operable communication with the database, the user dashboard to provide an interface for engaging with the asset information;
a calendar module to receive scheduling information corresponding to the plurality of asset information; and
an asset manager to receive the plurality of asset information and generate a heat map of the plurality of assets to facilitate the management of the plurality of assets.

2. The system of claim 1, wherein the plurality of asset information comprises at least one of the following: customer information, equipment information, inventory information, asset location information, service requests, preventative maintenance information, risk ratings, code references, and asset tag tracking information.

3. The system of claim 2, further comprising a plan of action system to receive the plurality of asset information and identify a deficiency with one or more assets.

4. The system of claim 3, wherein the plan of action system comprises a compliance engine to identify a deficiency in compliance.

5. The system of claim 4, wherein the plan of action system further comprises an action engine to identify an action to correct the deficiency in compliance.

6. The system of claim 5, wherein the plan of action system further comprises a quote generator to generate a quote corresponding to the action to correct the deficiency in compliance.

7. The system of claim 6, further comprising a service ticket generator to generate a service ticket corresponding to the quote.

8. The system of claim 7, wherein the quote generator is in operable communication with the calendar module to monitor a status of the quote.

9. The system of claim 8, wherein the quote generator is in operable communication with the calendar module to schedule one or more reminders corresponding to the quote, wherein the reminders are transmitted to at least one user.

10. A system for managing medical gasses systems, comprising:
a database configured to store a plurality of asset information;
a user dashboard provided on an interface of a computing device in operable communication with the database, the user dashboard to provide an interface for engaging with the asset information;
a calendar module to receive scheduling information corresponding to the plurality of asset information;
a quote generator to generate a quote via a plan of action system, the quote corresponding to an action to correct a deficiency in compliance, the quote generator in operable communication with the calendar module to monitor the status of the quote; and
an asset manager to receive the plurality of asset information and generate a heat map of the plurality of assets to facilitate the management of the plurality of assets.

11. The system of claim 10, further comprising a preventative maintenance engine to schedule and manage preventative maintenance corresponding to the plurality of assets.

12. The system of claim 11, wherein the plurality of asset information comprises at least one of the following: customer information, equipment information, inventory information, asset location information, service requests, preventative maintenance information, risk ratings, code references, and asset tag tracking information.

13. The system of claim 12, wherein the preventative maintenance engine generates an air compressor maintenance report and a vacuum maintenance report.

14. The system of claim 13, wherein the preventative maintenance engine generates a manufacturer maintenance report corresponding to one or more of the plurality of assets.

15. The system of claim 13, wherein the plan of action system to receive the plurality of asset information and identify a deficiency with one or more assets.

16. The system of claim 14, wherein the plan of action system comprises a compliance engine to identify a deficiency in compliance.

17. The system of claim 15, wherein the plan of action system further comprises an action engine to identify an action to correct the deficiency in compliance.

18. The system of claim 16, further comprising a service ticket generator to generate a service ticket corresponding to the quote.

19. The system of claim 17, wherein the quote generator is in operable communication with the calendar module to schedule one or more reminders corresponding to the quote, wherein the reminders are transmitted to at least one user.

20. A system for managing medical gasses systems, comprising:
a database configured to store a plurality of asset information;
a user dashboard provided on an interface of a computing device in operable communication with the database, the user dashboard to provide an interface for engaging with the asset information;
a calendar module to receive scheduling information corresponding to the plurality of asset information;
a quote generator to generate a quote via a plan of action system, the quote corresponding to an action to correct a deficiency in compliance, the quote generator in operable communication with the calendar module to monitor the status of the quote, the plan of action system to identify a compliance deficiency and determine a corrective action to correct the compliance deficiency; and
an asset manager to receive the plurality of asset information and generate a heat map of the plurality of assets to facilitate the management of the plurality of assets.
